(19) United States
(12) Patent Application Publication
(10) Pub. No.: US 2025/0296908 A1
(43) Pub. Date: Sep. 25, 2025

(54) METHOD FOR SEPARATING LEVULINIC ACID BY THERMAL SEPARATION IN THE PRESENCE OF A FLUX

(71) Applicant: IFP Energies nouvelles, Rueil-Malmaison Cedex (FR)

(72) Inventors: Pierre-Alain BREUIL, Rueil-Malmaison Cedex (FR); Maria FERNANDEZ ESPADA PASTOR, Rueil-Malmaison Cedex (FR)

(73) Assignee: IFP Energies nouvelles, Rueil-Malmaison (FR)

(21) Appl. No.: 18/869,545

(22) PCT Filed: May 11, 2023
(86) PCT No.: PCT/EP2023/062613

§ 371 Date: Nov. 26, 2024

(30) Foreign Application Priority Data
May 31, 2022 (FR) 2205179

(51) Int. Cl. C07C 51/44; B01D 1/22

(52) U.S. Cl. CPC C07C 51/44; B01D 1/22

(57) ABSTRACT

The present invention relates to a process for separating levulinic acid from a composition comprising levulinic acid and humins, wherein said composition is subjected to a step of thermal separation in the presence of a flux having a boiling point greater than that of the levulinic acid, so as to obtain a light fraction containing the levulinic acid and a heavy fraction containing the humins and said flux. The presence of a flux makes it possible to reduce the viscosity of the humins and to increase the recovery rate of levulinic acid.

### TECHNICAL FIELD

The present invention relates to a process for separating levulinic acid contained in a reaction medium resulting from the synthesis of levulinic acid. More particularly, the present invention relates to a process for separating levulinic acid involving a step of thermal separation in the presence of a flux.

### PRIOR ART

Levulinic acid (also known as 4-oxopentanoic acid or γ-ketovaleric acid) is an organic product corresponding to the formula:

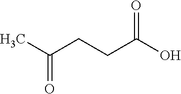

Levulinic acid is a product or a chemical intermediate that may be used in the petrochemical industry, the refining of petroleum products, the agricultural industry, the pharmaceutical industry, the food industry, the hygiene and cosmetics industry or also in the polymers and additives industry.

Levulinic acid is generally produced in two ways. The first route is the hydration of furfuryl alcohol in the presence of a homogeneous or heterogeneous acid catalyst. This synthesis is described for example in FR2640263, U.S. Pat. Nos. 3,752,849 and 2,780,588.

The second route, the sugar/biomass route, is the production of levulinic acid by acid hydrolysis starting from C6 or C5 sugars which may themselves be obtained from lignocellulosic biomass by acid hydrolysis, as described for example in Biofuels, Bioproducts and Biorefining 5 198-214 (2011). In addition to levulinic acid, the biomass or sugar hydrolyzates generally also contain compounds having a low boiling point, such as formic acid, acetic acid and propionic acid.

However, the yield of such processes, whether these be via the hydration of furfuryl alcohol route or the sugar/biomass route, is in fact rather low, mainly because of the formation of numerous reaction byproducts, from which the levulinic acid must be separated by complex separation and purification processes. In addition to various low-molecular-weight byproducts, the thermal treatment at acidic pH under stringent conditions leads to the formation of humins, which are high-molecular-weight polymeric compounds resulting from condensation reactions. Humins are generally separated in the form of solids, generally of a dark colour, which present a number of problems during the process of recovering the levulinic acid, notably via fouling of the equipment which can lead to complete clogging.

Another problem is the viscosity of the humins, which increases as a function of the heating time during a thermal separation step, which contributes to significant fouling of the separation equipment and/or to degrading the capacity for recovering the levulinic acid.

Another problem is the heat sensitivity of the levulinic acid itself, which is converted in a thermal separation step such as a distillation into undesired byproducts, for example by dehydration of the levulinic acid into angelica lactone. Such conversions lower the recovery rate of levulinic acid.

The main barrier in the obtaining of levulinic acid therefore seems to be less the synthesis and more its separation from the reaction medium and its purification. The separation and purification methods commonly used for separating levulinic acid from the reaction medium comprise solvent extraction, vacuum distillation, crystallization, ion exchange, membrane separation, etc. Such separation methods are described for example in WO2012/065115, WO2012/162028, WO2015/007602 or also CN107867996. Such separation methods have the disadvantage of being complicated to implement and are generally expensive.

There are also separation processes based solely on thermal separation steps, such as distillation or evaporation. Document WO2018/235012 for example describes a separation and purification method involving two distillation steps.

The present invention is directed to separating levulinic acid from the humins formed during the synthesis of levulinic acid, in particular by a step of thermal separation in the presence of a flux.

### SUMMARY OF THE INVENTION

More precisely, the invention relates to a process for separating levulinic acid from a composition comprising levulinic acid and humins and optionally compounds having a boiling point lower than that of the levulinic acid, wherein said composition is subjected to a step of thermal separation in the presence of a flux having a boiling point greater than that of the levulinic acid, so as to obtain a light fraction containing the levulinic acid and a heavy fraction containing the humins and said flux.

The present invention relates in particular to the act of using a flux during the thermal separation step which makes it possible to separate the levulinic acid from the humins formed in particular during the synthesis of the levulinic acid. The use of a flux makes it possible to significantly improve the recovery rate (yield) of levulinic acid compared to conditions in which the flux is not used.

Moreover, the use of a flux also makes it possible to control the viscosity of the humins, in particular by reducing their viscosity. Indeed, in the absence of a flux, the humins are often recovered as a solid at ambient temperature. The presence of a flux makes it possible to recover a heavy fraction containing the humins in liquid and viscous form at ambient temperature, thus facilitating the discharge thereof in the separation unit and therefore limiting fouling of the equipment.

The present invention therefore relates to a process for separating levulinic acid from a composition comprising levulinic acid and humins which makes it possible to simultaneously increase the recovery rate of levulinic acid while controlling the viscosity of the residue formed.

According to a variant, the flux is mixed with said composition and the amount of flux introduced into said mixture is such that the content by mass of the flux in said mixture is between 0.5% and 85% by weight relative to the weight of the mixture.

According to a variant, the thermal separation temperature is between 8° and 200° C. and the pressure is between 0.0001 and 0.1 MPa.

According to a variant, the flux has a boiling range of between 25° and 620° C. and is of petroleum origin and/or of vegetable origin and/or based on polymers or a mixture thereof.

According to a variant, the flux is chosen from a petroleum cut chosen from a vacuum gas oil, a heavy oil obtained from a fluidized-bed catalytic cracking, a settling oil, an unconverted oil originating from a hydrocracker, or a polyethylene glycol having an average molar mass of greater than or equal to 600 g/mol.

According to a variant, the separation step is carried out in at least one distillation column and/or in at least one evaporator.

According to a variant, the evaporator is a thin film evaporator.

According to a variant, when the composition comprising levulinic acid and humins comprises compounds having a boiling point lower than that of the levulinic acid, said composition is subjected to a step of preliminary thermal separation so as to separate off the compounds having a boiling point lower than that of the levulinic acid.

According to this variant, the thermal separation temperature is between 25 and 200° C. and the pressure is between 0.0001 and 0.2 MPa.

According to this variant, the separation step is carried out in at least one distillation column and/or in at least one evaporator.

According to a variant, the composition comprising levulinic acid and humins is obtained from the synthesis of levulinic acid by hydration of furfuryl alcohol in the presence of an acid catalyst and a solvent.

According to a variant, the acid catalyst is hydrochloric acid and the solvent is methyl ethyl ketone and/or 1,4-dioxane and/or 1,2-dimethoxyethane.

According to a variant, the composition comprising levulinic acid and humins is obtained from the synthesis of levulinic acid by acid hydrolysis of sugar and/or of biomass.

In the present description, the term “comprise” is synonymous with (means the same thing as) “include” and “contain”, and is inclusive or open-ended and does not exclude other elements which are not mentioned. It is understood that the term “to comprise” includes the exclusive and closed term “to consist of”.

In the present description, the expression “of between . . . and . . . ” means that the limiting values of the interval are included in the described range of values, unless specified otherwise.

In the present invention, the different ranges of values of given parameters can be used alone or in combination. For example, a preferred range of pressure values can be combined with a more preferred range of temperature values, or a preferred range of values for one chemical compound or element can be combined with a more preferred range of values for another chemical compound or element.

Hereinafter, particular and/or preferred embodiments of the invention may be described.

They can be employed separately or combined together, without limitation of combination when this is technically feasible.

In the present invention, the boiling temperature is measured under standard conditions, namely at 1 atmosphere, or 760.00 mmHg. At this pressure, the boiling temperature of pure water is 100° C. and the boiling point of levulinic acid is 245° C.

### DETAILED DESCRIPTION

### Synthesis of Levulinic Acid

The levulinic acid may be synthesized by any method known to a person skilled in the art. It is generally produced in two ways.

According to a first variant, the levulinic acid may be synthesized by hydration of furfuryl alcohol in the presence of a homogeneous or heterogeneous acid catalyst according to the following formula:

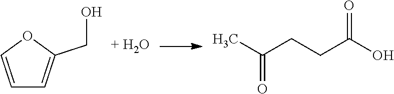

Levulinic acid is synthesized by heating the alcohol in a continuously operating or non-continuously operating reactor, in the presence of water and an acid catalyst and optionally a solvent.

The synthesis by hydration of furfuryl alcohol may be implemented in a continuously operating or non-continuously operating unit.

When the synthesis is implemented in a continuously operating unit, the furfuryl alcohol is introduced into the reactor by pouring, by injection or by any other means, on the one hand, and the solvent, water and acid mixture is introduced by pouring, by injection or by any other means, on the other hand, taking into account a target residence time. The withdrawal of the reaction effluent containing the levulinic acid formed is carried out continuously at the same time.

The synthesis by hydration of furfuryl alcohol may also be implemented in a unit operating as a reactor that is continuously fed, over the course of which no withdrawal of the contents of the reactor is carried out, i.e. in “fed batch” mode.

In the case of a fed-batch mode, the furfuryl alcohol is introduced into the reactor continuously, by pouring, by injection or by any other means, into the unit containing the water, the acid catalyst and the solvent. The reaction medium may be stirred. At the end of the reaction, the reaction effluent containing the levulinic acid formed is sent continuously into a separation section as described below.

The synthesis by hydration of furfuryl alcohol may also be implemented in a unit operating as a closed reactor, i.e. in “batch” mode.

In the case of a batch mode, all of the compounds (furfuryl alcohol, water, solvent, acid catalyst) are placed in a reactor, and then the reaction is carried out while heating. At the end of the reaction, the reaction effluent containing the levulinic acid formed is sent into a separation section as described below.

In the case of continuous operation, the compound (i.e. water or acid or solvent) to furfuryl alcohol molar ratio corresponds to the molar flow rate of said compound entering the reactor in relation to the molar flow rate of furfuryl alcohol entering the reactor.

In the case of fed-batch operation, the compound (i.e. water or acid or solvent) to furfuryl alcohol molar ratio corresponds to the total amount of said compound introduced into the reactor during the whole of the reaction in relation to the total amount of furfuryl alcohol introduced into the reactor during the whole of the reaction.

Independently of the amounts of material used for the synthesis, the duration of addition corresponds to the duration over which the furfuryl alcohol is introduced into the reaction section. This addition may be performed continuously or batchwise. The duration of addition is generally between 5 minutes and 4 days, preferably between 1 hour and 2 days, very preferably between 2 hours and 1 day.

At the end of the reaction the reaction effluent may be stirred under the temperature and pressure conditions of the reaction for a maturation phase. The maturation phase is generally between 1 second and 4 days, preferably between 1 minute and 2 days, very preferably between 5 minutes and 1 day. At the end of this maturation phase, the reaction effluent containing the levulinic acid formed may be sent into a separation section as described below.

The furfuryl alcohol may be biobased or non-biobased. It may, for example, be obtained from C5 sugars (comprising 5 carbon atoms) or C6 sugars (comprising 6 carbon atoms).

The water is usually present in an amount such that the water/furfuryl alcohol molar ratio is between 0.9 and 10.0 mol/mol, preferably between 1.0 and 5.0 mol/mol, very preferably between 1.1 and 3.0 mol/mol.

According to the invention, the process is implemented in the presence of at least one catalyst chosen from homogeneous or heterogeneous and organic or inorganic Brønsted acids.

In one embodiment, at least one catalyst is chosen from homogeneous or heterogeneous organic Brønsted acids.

Preferably, the homogeneous organic Brønsted acid catalysts are chosen from organic acids of general formulae R′COOH, R′SO₂H, R′SO₃H, (R′SO₂)NH, (R′O)₂PO₂H, R′OH, in which R′ is chosen from the following groups:
alkyls, preferably comprising between 1 and 15 carbon atoms, preferably between 1 and 10 and preferably between 1 and 6, which are or are not substituted by at least one substituent chosen from a hydroxyl, an amine, a nitro, a halogen, preferably fluorine, and an alkyl halide,
alkenyls, which are or are not substituted by at least one group chosen from a hydroxyl, an amine, a nitro, an oxo, a halogen, preferably fluorine, and an alkyl halide,
aryls comprising between 5 and 15 carbon atoms and preferably between 6 and 12 carbon atoms, which are or are not substituted by a substituent chosen from a hydroxyl, an amine, a nitro, an oxo, a halogen, preferably fluorine, and an alkyl halide,
heteroaryls comprising between 4 and 15 carbon atoms and preferably between 4 and 12 carbon atoms, which are or are not substituted by a substituent chosen from a hydroxyl, an acid, an amine, a nitro, an oxo, a halogen, preferably fluorine, and an alkyl halide.

When the catalysts of organic Brønsted acid type are chosen from organic acids of general formula R′—COOH, R′ may also be a hydrogen.

Preferably, the organic Brønsted acids are chosen from formic acid, acetic acid, trifluoroacetic acid, lactic acid, levulinic acid, 2,5-furandicarboxylic acid, methanesulfinic acid, methanesulfonic acid, trifluoromethanesulfonic acid, bis(trifluoromethanesulfonyl)amine, benzoic acid, para-toluenesulfonic acid, 4-biphenylsulfonic acid, diphenyl phosphate and 1,1′-binaphthyl-2,2′-diyl hydrogen phosphate. Very preferably, the homogeneous organic Brønsted acid catalyst is chosen from methanesulfonic acid (CH₃SO₃H), para-toluenesulfonic acid and trifluoromethanesulfonic acid (CF₃SO₃H).

The heterogeneous organic Brønsted acid catalysts are chosen from ion-exchange resins, in particular from sulfonic acid resins based on a copolymer preferably of sulfonated styrene/divinylbenzene or on a sulfonated tetrafluoroethylene copolymer (such as, for example, the following commercial resins: Amberlyst® 15, 16, 35 or 36, Dowex® 50 WX2, WX4 or WX8, Nafion® PFSA NR-40 or NR-50, or Aquivion® PFSA PW 66, 87 or 98), charcoals functionalized by sulfonic and/or carboxylic groups, or silicas functionalized by sulfonic and/or carboxylic groups. Preferably, the heterogeneous organic Brønsted acid catalyst is chosen from sulfonic acid resins.

In one embodiment, at least one catalyst is chosen from homogeneous or heterogeneous inorganic Brønsted acids.

Preferably, the homogeneous inorganic Brønsted catalysts are chosen from HF, HCl, HBr, HI, H₂SO₃, H₂SO₄, H₃PO₂, H₃PO₄, HNO₂, HNO₃, H₂WO₄, H₄SiW₁₂O₄₀, H₃PW₁₂O₄₀, (NH₄)₆(W₁₂O₄₀)·XH₂O, H₄SiMo₁₂O₄₀, H₃PMo₁₂O₄₀, (NH₄)₆Mo₇O₂₄·xH₂O, H₂MoO₄, HReO₄, H₂CrO₄, H₂SnO₃, H₄SiO₄, H₃BO₃, HClO₄, HBF₄, HSbF₅, HPF₆, H₂FO₃P, CISO₃H, FSO₃H, HN(SO₂F)₂ and HIO₃. Preferably, the inorganic Brønsted acids are chosen from HCl, HBr, HI, H₂SO₄, H₃PO₄ or HNO₃. Very preferably, the inorganic Brønsted acid is HCl.

Preferably, the heterogeneous inorganic Brønsted catalysts are chosen from oxides, hydroxides or phosphates of the elements of columns 1 to 14 of the periodic table of the elements, taken alone or as a mixture. Preferably, the heterogeneous inorganic catalyst is chosen from crystalline or non-crystalline aluminosilicates and crystalline or non-crystalline aluminosilicophosphates. Very preferably, the heterogeneous inorganic catalyst is chosen from materials from the class of the zeolites.

The acid catalyst is usually present in an amount such that the acid/furfuryl alcohol molar ratio is between 0.01 and 1.0 mol/mol, preferably between 0.02 and 0.5 mol/mol.

The synthesis by hydration of furfuryl alcohol may be carried out in the presence of a solvent. The solvent is preferably a solvent in which the water is partly or completely soluble. The solvent advantageously has a lower boiling point than that of levulinic acid, which makes it possible to separate the solvent for recycling and to limit the heating temperature when the optional preliminary thermal separation step is carried out, thus avoiding the formation of humins and/or the degradation of the levulinic acid.

According to a variant, the solvent may be chosen from an aliphatic ketone, such as acetone, methyl ethyl ketone, diethyl ketone, methyl isobutyl ketone, methyl isopropyl ketone or cyclohexanone. Preferably, the solvent is methyl ethyl ketone.

According to another variant, the solvent may be chosen from an ether and/or an acetal. According to this variant, the solvent is chosen from the compounds corresponding to one or the other of the structures I and II, taken alone or as a mixture:

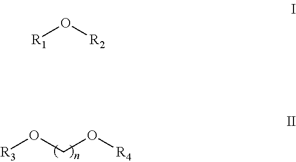

in which R₁, R₂, R₃ and R₄ are independently chosen from:
   linear or branched aliphatic groups of 1 to 6 carbon atoms, optionally substituted by alkoxy groups,
   cyclic or polycyclic aliphatic groups of 5 to 12 carbon atoms, optionally substituted by alkoxy or alkyl groups,
   linear or branched olefinic groups of 1 to 6 carbon atoms, optionally substituted by alkoxy groups,
   aromatic or polyaromatic groups of 6 to 12 carbon atoms,
R₁ and R₂ may be bonded together by covalent bonds so as to form a ring,
R₃ and R₄ may be bonded together by covalent bonds so as to form a ring,
n is an integer between 1 and 6.

According to a variant, the solvent is an ether-based solvent and is chosen from diethyl ether, diisopropyl ether, diisobutyl ether, dibutyl ether, diphenyl ether, 2-methoxy-2-methylpropane, 2-methoxy-2-methylbutane, 2,5-dihydrofuran, tetrahydrofuran, 2-methyltetrahydrofuran, 3-methyltetrahydrofuran, 2,3-dihydropyran, tetrahydropyran, 1,3-dioxolane, 1,3-dioxane, 1,4-dioxane, 1,2-dimethoxyethane and benzofuran, taken alone or as a mixture.

According to another variant, the solvent is an acetal-based solvent and is chosen from 2,2-dimethoxypropane, 2,2-di(2-ethylhexyloxy)propane, 2-methoxytetrahydrofuran and di(2-methoxyethyl)ether, taken alone or as a mixture.

According to another variant, the solvent is a mixture of an ether- and acetal-based solvent, chosen from one of the ethers and acetals mentioned above, in any proportion.

Preferably, the solvent is chosen from diisobutyl ether, dibutyl ether, 2-methyltetrahydrofuran, 3-methyltetrahydrofuran, 1,4-dioxane and 1,2-dimethoxyethane, taken alone or as a mixture.

Even more preferably, the solvent is chosen from 1,4-dioxane and 1,2-dimethoxyethane, taken alone or as a mixture.

According to another variant, the solvent may be a mixture of an aliphatic ketone and an ether and/or an acetal, in any proportion.

Preferably, the synthesis by hydration of furfuryl alcohol is carried out in the presence of a solvent, and preferably in the presence of methyl ethyl ketone and/or 1,4-dioxane and/or dimethoxyethane.

Preferably, the levulinic acid is synthesized by hydration of furfuryl alcohol in the presence of a homogeneous acid catalyst, preferably hydrochloric acid, and of a solvent, preferably methyl ethyl ketone and/or 1,4-dioxane and/or 1,2-dimethoxyethane.

The solvent is usually present in an amount such that the solvent/furfuryl alcohol molar ratio is between 0.1 and 5 mol/mol, preferably between 0.5 and 3 mol/mol, very preferably between 1 and 2 mol/mol.

The synthesis by hydration of furfuryl alcohol is generally carried out at a temperature of between 25 and 140° C., preferably of between 4° and 120° C., very preferably of between 6° and 110° C.

The synthesis by hydration of furfuryl alcohol is generally carried out at a pressure of between 0.01 MPa and 1 MPa (0.1 bara and 10 bara), and preferably at atmospheric pressure.

The conversion of the furfuryl alcohol is generally greater than 95%, preferably greater than 98%, very preferably greater than 99%.

At the end of the synthesis by hydration of furfuryl alcohol, a reaction effluent is obtained containing levulinic acid, water, acid catalyst (preferably hydrochloric acid HCl), solvent (preferably methyl ethyl ketone and/or 1,4-dioxane and/or 1,2-dimethoxyethane), and possibly traces of unconverted furfuryl alcohol, and also the unavoidable humins formed which are high-molecular-weight products resulting from condensation reactions, in particular by condensation of furfuryl alcohol with itself. The humins are soluble in the reaction medium.

According to a second variant, the levulinic acid may be synthesized from sugar and/or biomass by acid hydrolysis.

The term “biomass” refers to a material derived from recently living organisms, which comprises plants, animals and by-products thereof. The term “lignocellulosic biomass” denotes biomass derived from plants or from by-products thereof. The lignocellulosic biomass is composed of carbohydrate polymers (cellulose, hemicellulose) and of an aromatic polymer (lignin). The biomass may for example be derived from grass, cereals, starch, algae, tree bark, hay, straw, leaves, paper pulp, paper sludge or manure. The biomass, and in particular the cellulose contained in the biomass, is converted by acid hydrolysis to C6 sugars (glucose and/or fructose).

The levulinic acid may also be synthesized by acid hydrolysis of C6 sugars, in particular fructose or glucose or mixtures thereof. Sucrose can be broken down into a glucose molecule plus a fructose molecule in a weakly acidic environment by a process called inversion. Fructose can also be manufactured by enzymatic isomerization of glucose. Sucrose is commonly produced from biomass such as beet, corn and sugar cane.

For example, proceeding from sucrose, the reaction scheme that leads to the formation of levulinic acid is as follows (HMF=2,5-(hydroxymethyl) furaldehyde or 2,5-(hydroxymethyl) furfural):

sucrose (C₁₂H₂₂O₁₁)+H₂O-->glucose (C₆H₁₂O₆)+fructose (C₆H₁₂O₆) (I)

fructose (C₆H₁₂O₆)->HMF (C₆H₆O₃)+3 H₂O (II)

HMF (C₆H₆O₃)+2 H₂O->levulinic acid (C₅H₈O₃)+formic acid (CH₂O₂) (III)

The acid hydrolysis of biomass or sugar is carried out in the presence of an acid. Suitable acids include sulfuric acid, hydrochloric acid and phosphoric acid. A preferred acid is sulfuric acid, preferably dilute sulfuric acid, for example at a concentration of between 1.5% and 3%.

The temperature in the acid hydrolysis is generally between 15° and 250° C., preferably between 17° and 240° C., more preferably between 19° and 230° C., and more preferably still between 20° and 220° C.

The pressure is generally between 0.1 and 5 MPa, preferably between 0.5 and MPa, and more preferentially still between 1 and 3 MPa.

The acid hydrolysis may comprise one, two or more steps. Suitable reactors include, for example, plug-flow reactors or CSTR (continuous stirred-tank reactor) reactors. It is possible to use different reactors for different steps.

The acid hydrolysis of biomass and/or sugar not only results in the formation of levulinic acid, but generally also in the formation of compounds having a boiling point lower than that of the levulinic acid, such as formic acid, acetic acid, furfural and propionic acid. In addition, humins are formed. At the end of the synthesis by acid hydrolysis of biomass and/or sugar, a reaction effluent is thus obtained containing levulinic acid, water, acid and compounds having a boiling point lower than that of the levulinic acid, and also the unavoidable humins.

This reaction effluent may be subjected directly to a thermal separation step or may undergo one or more additional steps. Examples of such steps include solid/liquid separation and/or liquid-liquid extraction using a water-immiscible solvent. A combination of two or more of these steps is also possible.

Suitable solid-liquid separation techniques include filtration and centrifugation.

The liquid-liquid extraction is carried out by mixing the aqueous solution comprising the levulinic acid resulting from the acid hydrolysis of sugar and/or biomass with a water-immiscible solvent in order to give an organic phase comprising recovered levulinic acid (and soluble humins) and an aqueous phase in which a portion of the residues remains. Examples of water-immiscible solvents suitable as extraction solvents are ketones, ethers or acetates of low molecular weight, such as those containing more than five carbon atoms, for example solvents derived from furan. The water-immiscible solvent has a lower boiling point than levulinic acid. The water-immiscible solvent may for example be chosen from furfural, hydroxymethylfurfural, gamma-valerolactone (GVL), methyl isobutyl ketone (MIBK), methyltetrahydrofuran (MTHF) and combinations thereof. The liquid-liquid extraction is generally carried out at a temperature of between 2° and 100° C., preferably at ambient temperature. The water-immiscible solvent/acid hydrolyzate ratio is generally between 0.25:1 and 5:1, more preferentially between 1:1 and 4:1. The liquid-liquid extraction is carried out with the aid of means capable of extraction, preferably extraction columns, centrifugal extractors, or a mixer-decanter apparatus.

In order to obtain the levulinic acid in high yield and high purity, the reaction effluent, whether this be obtained from the hydration of furfuryl alcohol or obtained directly from the acid hydrolysis of biomass and/or sugar, or else obtained from the liquid/liquid extraction of the acid hydrolyzate obtained from biomass and/or sugar and containing a water-immiscible solvent, must be subjected to a separation process.

### Preliminary Thermal Separation Step (Optional)

Before carrying out the step of thermal separation in the presence of a flux according to the present invention, the reaction effluent may be subjected to a preliminary thermal separation step aimed at separating off the compounds having a boiling point lower than that of the levulinic acid.

The preliminary thermal separation step may be carried out according to any method known to a person skilled in the art. It may, for example, be carried out by distillation and/or by evaporation.

According to a variant, and when it is carried out by distillation, use may be made of a plate distillation column. The number of theoretical plates is generally between 1 and 50, preferably between 1 and 10.

The column-bottom distillation temperature is advantageously between 25 and 200° C., preferably between 5° and 180° C., and very preferably between 10° and 160° C.

The column-top distillation pressure is advantageously between 0.0001 and 0.2 MPa (between 1 mbara and 2 bara), preferably between 0.001 and 0.1 MPa (between 10 mbara and 1 bara), and very preferably between 0.004 and 0.05 MPa (between 40 mbara and 500 mbara).

According to another variant, and when the preliminary thermal separation step is carried out by distillation, use may also be made of a packed distillation column operating within the same temperature and pressure ranges.

According to another variant, and when the preliminary thermal separation step is carried out by evaporation, use may be made of one or more evaporators in series or in parallel; the evaporator(s) may be chosen for example from natural or forced circulation evaporators, falling or climbing film evaporators, agitated thin film evaporators, plate evaporators or multiple-effect evaporators. Preferably, at least two evaporators are used in series. Falling film or climbing film evaporators are known devices in which the heating and the conversion of the liquid to vapor are carried out within a plurality of tubes, themselves heated by a fluid (for example low-pressure steam), inside which the liquid flows in the form of a film along the inner wall of the tubes. The heat applied through the walls of each tube causes the light fraction of the liquid mixture to evaporate. In the case of a falling film evaporator, the film of liquid flows downwards, by virtue of the action of the force of gravity, whereas in the case of a rising film evaporator, the liquid film is pushed upwards by the vapor generated from the boiling. In this way, the liquid is heated rapidly, with high-temperature residence times that are quite reduced compared to distillation, and consequently a lower risk of degradation of the organic products present in the liquid itself.

The evaporator(s) operate(s) within the same temperature and pressure ranges as described for the distillation.

The preliminary thermal separation step aims in particular to separate the compounds having a boiling point lower than that of the levulinic acid from the reaction effluent, making it possible to obtain said composition comprising levulinic acid and humins freed of the light compounds, which is at least in part, continuously or batchwise, introduced into the separation process according to the invention including the step of separation in the presence of the flux.

The preliminary thermal separation step may be carried out in the absence or in the presence of a flux as defined below, and preferably it is carried out in the absence of a flux.

In the case of the reaction effluent resulting from the hydration of furfuryl alcohol, the preliminary thermal separation step makes it possible to separate off a light fraction comprising the water, the acid (in particular hydrochloric acid) and the solvent (in particular methyl ethyl ketone and/or 1,4-dioxane and/or 1,2-dimethoxyethane). These compounds are preferably then condensed and recycled into the synthesis unit, which makes it possible firstly to limit the emissions and the environmental impact of the synthesis and secondly to limit the consumption of resources and ultimately the production cost of the levulinic acid.

In the case of the reaction effluent resulting from the acid hydrolysis of biomass and/or sugar, the preliminary thermal separation step makes it possible to separate off a light fraction comprising the water, the acid and the compounds having a boiling point lower than that of the levulinic acid, such as formic acid, acetic acid, furfural and propionic acid. These compounds may be upgraded as chemical products or intermediates, after optional additional separation steps.

In the case of the reaction effluent resulting from a liquid/liquid extraction of acid hydrolyzate derived from biomass and/or sugar, the preliminary thermal separation step makes it possible to separate off a light fraction comprising the water-immiscible solvent.

The preliminary thermal separation step is generally carried out such that the content of water in the composition comprising levulinic acid and humins which is sent into the thermal separation step is less than 1% by weight relative to the total weight of the composition, preferably less than 0.9% by weight and particularly preferably less than 0.8% by weight.

### Separation Process Including a Step of Thermal Separation in the Presence of a Flux

The composition comprising levulinic acid and humins which has optionally been freed of compounds having a boiling point lower than that of the levulinic acid by the preliminary thermal separation step is then subjected to the separation process according to the invention and including a step of thermal separation in the presence of a flux having a boiling point greater than that of the levulinic acid, so as to obtain a light fraction containing the levulinic acid and a heavy fraction containing the humins and said flux.

Preferably, the composition comprising levulinic acid and humins no longer contains compounds having a boiling point lower than that of the levulinic acid during this step.

The flux should have a boiling point greater than that of the levulinic acid, which is 245° C., in order to be able to carry out the separation. The flux generally has a boiling point of greater than 250° C., preferably of greater than 280° C., and particularly preferably of greater than 300° C.

The flux should also be stable and should not degrade at a temperature of between 2° and 200° C., preferably between 15° and 200° C. Specifically, the flux should not degrade into compounds that might react with the levulinic acid or into lighter compounds that might be separated off with the light fraction containing the levulinic acid. The flux may contain polar or protic functions.

The flux may be of petroleum origin and/or of vegetable origin and/or based on polymers. The flux may also be a mixture of at least two of these components.

When the flux is of petroleum origin, it may be chosen from any petroleum cut having a boiling point greater than that of the levulinic acid, in particular from a vacuum gas oil (VGO) (which typically has a boiling range of from 360° C. to 620° C.), a settling oil or a recycle oil (which typically has a boiling range of from 360° C. to 620° C.), for example a fluidized-bed catalytic cracking effluent such as a heavy cycle oil (HCO), an unconverted oil originating from a hydrocracker which typically has a boiling range of from 360° C. to 620° C. (UCO), vacuum residues (which typically have a boiling range of greater than or equal to 524° C.), deasphalted oils, resins, or a mixture thereof.

When the flux is of vegetable origin, it may be chosen from a vegetable and/or animal oil or also from fatty acid methyl esters (FAMEs) which may be produced either by esterification of fatty acids derived from vegetable and/or animal oil or by direct transesterification of vegetable and/or animal oil, or a mixture thereof. Mention may for example be made of olive oils or avocado oil. This nonlimiting list also includes all oils obtained by genetic modification or hybridization. Spent oils, such as frying oils, and also all spent oils and fats from the catering industries, may also be used. With regard to animal fats, mention may be made, without being limiting, of tallow. The expressions “animal fat” and “animal oil” are used without distinction in the present description, the only difference between a fat and an oil being the state of the fatty substance at ambient temperature: liquid for an oil and solid for a fat.

These vegetable and/or animal oils may be crude or totally or partially refined. Typically, the distinction between a crude or a refined vegetable oil refers to the method by which it was extracted, mainly with pressing for a crude oil (typically a single pressing under cold conditions without additives) and generally using a solvent for a refined oil.

When the flux is based on polymers, it may be chosen from polyethers of the type of polyethylene glycol having an average molar mass of greater than or equal to 600 g/mol, and in particular PEG-600, PEG-800 or PEG 1000, or also PEG 6000 or PEG 8000, alone or as a mixture.

The flux is advantageously mixed with said composition before and/or during the performance of the thermal separation step. The mixing may be carried out before the separation unit or in the separation unit.

The mixing may be carried out by any means known to a person skilled in the art, for example a stirrer, recirculation of the liquid with a pump or by passing the mixture through a static mixer.

The amount of flux introduced into said mixture is such that the content by mass of the flux in said mixture is between 0.5% and 85% by weight, preferably between 1% and 70% by weight, and with preference between 1.5% and 50% by weight, and particularly preferably between 1% and 20% by weight, relative to the total weight of the mixture.

The mixing is advantageously carried out at a temperature of between 25 and 200° C., preferably of between 5° and 195° C., and very preferably of between 11° and 190° C.

The mixing time is advantageously between 0.1 and 600 minutes, preferably between 1 and 60 minutes.

In general, the flux makes it possible to reduce the viscosity of the humins contained in the heavy fraction. Indeed, in the absence of a flux, the heavy fraction containing the humins is often recovered as a solid at ambient temperature. The presence of a flux makes it possible to recover the heavy fraction in liquid and viscous form at ambient temperature, thus facilitating the discharge thereof in the separation unit and therefore limiting fouling of the equipment.

The flux also makes it possible to control the thermal separation of the levulinic acid by limiting the separation temperature and thus the formation of undesired secondary products of levulinic acid, such as angelica lactone. The presence of the flux thus makes it possible to significantly improve the recovery rate of levulinic acid via the conservation of the levulinic acid by limiting the separation temperature and by controlling the viscosity of the residue containing the humins, in particular by reducing its viscosity.

The step of thermal separation in the presence of the flux may be carried out according to any method known to a person skilled in the art. It may, for example, be carried out by distillation and/or by evaporation.

According to a variant, and when it is carried out by distillation, use may be made of a plate distillation column. The number of theoretical plates is generally between 1 and 50, preferably between 1 and 20.

The column-bottom distillation temperature is advantageously between 8° and 200° C., preferably between 10° and 195° C., and very preferably between 11° and 190° C.

The column-top distillation pressure is advantageously between 0.0001 and 0.1 MPa (between 1 mbara and 1 bara), preferably between 0.001 and 0.08 MPa (between 10 mbara and 800 mbara), and very preferably between 0.002 and 0.05 MPa (between 20 mbara and 500 mbara).

According to another variant, and when the step of thermal separation in the presence of the flux is carried out by distillation, use may also be made of a packed distillation column operating within the same temperature and pressure ranges.

According to another variant, and when the step of thermal separation in the presence of the flux is carried out by evaporation, use may be made of one or more evaporators in series or in parallel; the evaporator(s) may be chosen for example from natural or forced circulation evaporators, falling or climbing film evaporators, agitated thin film evaporators, plate evaporators or multiple-effect evaporators.

The evaporator(s) operate(s) within the same temperature and pressure ranges as described for the distillation.

Advantageously, when the step of thermal separation in the presence of the flux is carried out by evaporation, use may be made of a thin film evaporator.

In contrast to a falling or rising film evaporator, a thin film evaporator comprises a single tube inside of which the liquid to be treated flows along the inner wall of the tube itself. The film of liquid is distributed uniformly over the wall by virtue of the action of a blade rotor inserted within the tube which, when it is set in rotation, in addition to distributing the liquid over the wall, creates a turbulent flow within the film itself, which considerably improves the heat exchange. This type of evaporator makes it possible to rapidly separate the most volatile part from the least volatile part by virtue of the agitation of the liquid in the form of a film under controlled conditions. The evaporator preferably operates at reduced pressure in order to lower the temperature of separation of the vapor phase from the liquid phase. The heating of the wall of the tube is carried out for example by external coils within which circulates a heating fluid, for example steam.

This type of equipment makes it possible to very significantly limit the residence time of the products/residues compared to a distillation column or falling or rising film evaporators. Specifically, an increase in the viscosity of the residue as a function of the time at high temperature (150-200° C.) is observed, which contributes to significant fouling of the separation equipment. The use of a thin film evaporator makes it possible to limit the residence time of said composition comprising levulinic acid and humins and therefore to significantly limit the increase in viscosity and hence to improve the operability of this separation step.

The recovery rate of levulinic acid, corresponding to the mass of levulinic acid vaporized in relation to the mass of levulinic acid contained in the composition used in the step of separation in the presence of a flux, is generally between 80% and 99%, and preferably between 82% and 99%.

The obtained purity of levulinic acid is between 90.0% and 99.0% by weight, preferably between 90.1% and 98.0% by weight, very preferably between 90.2% and 97.9% by weight. If necessary, and in order to increase the purity, the levulinic acid may be subjected to one or more purification steps, such as a crystallization or esterification step.

The light fraction comprising the levulinic acid may then be cooled and condensed. The cooling and the condensation of this fraction may optionally be integrated into a production of low-pressure steam by heat exchange, thus enabling a saving of energy. The low-pressure steam may be used as heating in the evaporator(s).

The heavy fraction containing the humins and the flux is liquid and therefore easy to discharge from the separation unit. It may be sent to an external waste treatment. It may also be burned to produce thermal energy, for example for the separation unit(s).

According to a preferred variant, the process for separating levulinic acid is carried out starting from a composition comprising levulinic acid and humins and compounds having a boiling point lower than that of the levulinic acid, wherein the following steps are carried out:
said composition is subjected to a preliminary thermal separation step so as to separate off the compounds having a boiling point lower than that of the levulinic acid,
said composition comprising levulinic acid and humins is then subjected to a step of thermal separation in the presence of a flux having a boiling point greater than that of the levulinic acid, so as to obtain a light fraction containing the levulinic acid and a heavy fraction containing the humins and said flux.

### LIST OF THE FIGURES

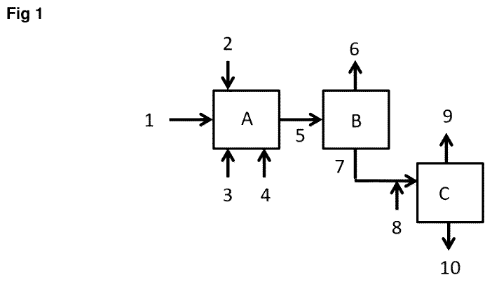

The information regarding the elements referenced in FIG. 1 enables a better understanding of the invention, without said invention being limited to the particular embodiments illustrated in FIG. 1. The various embodiments presented can be used alone or in combination with one another, without limitation of combination.

FIG. 1 represents the diagram of a preferred embodiment of the process of the present invention, comprising:
the synthesis of levulinic acid by hydration of furfuryl alcohol in the presence of an acid catalyst in a continuously operating or non-continuously operating reactor A in which furfuryl alcohol 1, water 2, hydrochloric acid 3 and the solvent methyl ethyl ketone (MEK) 4 are introduced and this mixture is heated in order to synthesize the levulinic acid. The reaction effluent 5 is sent continuously or batchwise into a preliminary thermal separation section B which can be implemented by distillation or evaporation. The preliminary thermal separation step B makes it possible to separate a light fraction 6 containing the hydrochloric acid, the solvent and the unconverted water, which can be at least partly recycled into the reactor A (recycle not shown), and a heavy fraction (residue) which is the composition 7 comprising the levulinic acid and the humins freed of light compounds. This composition is mixed with a flux 8 and then this mixture is sent into a thermal separation section C which can be implemented by distillation or evaporation and which makes it possible to obtain a light fraction containing the levulinic acid 9 and a heavy fraction 10 containing the humins and the flux.

### EXAMPLES

Levulinic acid is synthesized by hydration of furfuryl alcohol in the presence of hydrochloric acid and methyl ethyl ketone (MEK) at a temperature of 75° C., at reflux and at atmospheric pressure. A reaction effluent is obtained containing levulinic acid, water, hydrochloric acid HCl, methyl ethyl ketone solvent and humins, and is subjected to a step of preliminary separation by distillation.

### Preliminary Distillation of Light Compounds

Operating conditions of the step of preliminary distillation of light compounds:

The feedstock 5 resulting from the step of hydration of furfuryl alcohol in acidic medium contains 36.0% by weight of levulinic acid, 45% by weight of MEK, 5.3% by weight of water, 2.2% by weight of hydrochloric acid, i.e. a molar yield of levulinic acid relative to the furfuryl alcohol employed of 80%. Humins (polymeric compounds) which are soluble in the reaction medium were formed in an amount of 11.5% by weight.

300 g of feedstock 5 are placed in a 500 mL round-bottom flask equipped with a stirrer and a condenser in order to perform a step B of preliminary thermal separation of the light compounds (HCl, MEK and water). The medium is heated at a temperature of 125° C. under a pressure of 100 mbara (0.01 MPa).

During this batch distillation, the temperature of the vapors under 100 mbara (0.01 MPa) is between 33 and 56° C.

163.5 g of distillate is obtained after this step; its composition is as follows: 83.1% by weight of MEK, 12.8% by weight of water and 4.0% by weight of HCl. 136.5 g of residue 7 is obtained after this step; its composition is as follows: 0.5% by weight of water, 74.1% by weight of levulinic acid and 25.4% by weight of humins.

### Example 1 (Comparative Example): Implementation of the Process Including the Thermal Separation Step C without the Use of Flux

133 g of residue 7 produced according to the description above are placed in a 300 mL round-bottom flask equipped with a stirrer and a condenser in order to perform the step of thermal separation of the levulinic acid by distillation. The distillation is performed under vacuum. The distillation step is implemented at a column-bottom temperature of 180° C. and under a vacuum of 5 mbara (0.0005 MPa). The levulinic acid distillate 9 obtained at the end of this step has a mass of 37 g and has a composition of 90.9% by weight of levulinic acid. The residue 10 recovered has a mass of 94 g and contains 61.9% by weight of levulinic acid.

The recovery rate by mass of levulinic acid is 63.5%, corresponding to the mass of levulinic acid recovered in the distillate relative to the mass of levulinic acid involved in the residue obtained from the step of distillation of light compounds. The residue 10, which is solid at ambient temperature, has a non-measurable viscosity.

### Example 2 (According to the Invention): Implementation of the Process According to the Invention Including the Thermal Separation Step C with the Use of PEG 600 as Flux

15.0 g of the residue 7 produced according to the description above is mixed with 3.0 g of the flux 8 PEG 600 (initial boiling point 270° C., at atmospheric pressure), in order to pass into the distillation step. The proportion of flux in said mixture 7+8 is 16.7% by weight relative to the total weight of the mixture 7+8.

The residue/flux mixture 7+8 is sent to step C of thermal separation of the levulinic acid by distillation. The distillation is performed under vacuum. The distillation step is implemented at a column-bottom temperature of 180° C. and under a vacuum of 5 mbara (0.0005 MPa), so as to facilitate the evaporation of the levulinic acid. The levulinic acid distillate obtained at the end of this step has a mass of 11.2 g and has a composition of 91.6% by weight of levulinic acid. The residue 10 recovered has a mass of 7 g and contains 19.7% by weight of levulinic acid. The rate of recovery of levulinic acid is 88%, corresponding to the mass of levulinic acid recovered in the distillate relative to the mass of levulinic acid involved in the residue obtained from the step of distillation of light compounds. The residue 10 containing the humins and the flux is liquid and viscous at ambient temperature.

### Example 3 (According to the Invention): Implementation of the Process According to the Invention Including the Thermal Separation Step C with the Use of HCO as Flux

HCO (HCO for Heavy Cycle Oil) is a heavy cycle oil resulting from fluidized-bed catalytic cracking and has a boiling point range of between 317 and 570° C.

15.0 g of the residue 7 produced according to the description above is mixed with 3.2 g of the flux 8 HCO, in order to pass into the distillation step. The proportion of flux in said mixture 7+8 is 17.6% by weight relative to the total weight of the mixture.

The residue/flux mixture 7+8 is sent to step C of thermal separation of the levulinic acid by distillation. The distillation is performed under vacuum. The distillation step is implemented at a column-bottom temperature of 180° C. and under a vacuum of 5 mbara (0.0005 MPa), so as to facilitate the evaporation of the levulinic acid. The levulinic acid distillate obtained at the end of this step has a mass of 12.0 g and has a composition of 90.8% by weight of levulinic acid. The residue 10 recovered has a mass of 5.6 g and contains 13.7% by weight of levulinic acid. The rate of recovery of levulinic acid is 93%, corresponding to the mass of levulinic acid recovered in the distillate relative to the mass of levulinic acid involved in the residue obtained from the step of distillation of light compounds. The residue 10 containing the humins and the flux is liquid and viscous at ambient temperature.

### Example 4: Implementation of the Process According to the Invention Including the Thermal Separation Step C with the Use of VGO as Flux

VGO (VGO for Vacuum Gas Oil) is a vacuum gas oil and has a boiling point range of between 292 and 602° C.

15.0 g of the residue 7 produced according to the description above is mixed with 3.3 g of the flux 8 VGO, in order to pass into the distillation step. The proportion of flux in said mixture 7+8 is 18.0% by weight relative to the total weight of the mixture.

The residue/flux mixture 7+8 is sent to step C of thermal separation of the levulinic acid by distillation. The distillation is performed under vacuum. The distillation step is implemented at a column-bottom temperature of 180° C. and under a vacuum of 5 mbara (0.0005 MPa), so as to facilitate the evaporation of the levulinic acid. The levulinic acid distillate obtained at the end of this step has a mass of 12.2 g and has a composition of 95.4% by weight of levulinic acid. The residue 10 recovered has a mass of 6.0 g and contains less than 0.1% by weight of levulinic acid. The rate of recovery of levulinic acid is 99%, corresponding to the mass of levulinic acid recovered in the distillate relative to the mass of levulinic acid involved in the residue 7. The residue 10 containing the humins and the flux is liquid and viscous at ambient temperature.

### Example 5: Implementation of the Process According to the Invention Including the Thermal Separation Step C with the Use of VGO as Flux

The VGO is the same as in example 4.

10.2 g of the residue 7 produced according to the description above is mixed with 0.6 g of the flux 8 VGO, in order to pass into the distillation step. The proportion of flux in said mixture 7+8 is 5.6% by weight relative to the total weight of the mixture.

The residue/flux mixture 7+8 is sent to step C of thermal separation of the levulinic acid by distillation. The distillation is performed under vacuum. The distillation step is implemented at a column-bottom temperature of 180° C. and under a vacuum of 5 mbara (0.0005 MPa), so as to facilitate the evaporation of the levulinic acid. The levulinic acid distillate obtained at the end of this step has a mass of 8.0 g and has a composition of 94.0% by weight of levulinic acid. The residue 10 recovered has a mass of 7.9 g and contains 0.8% by weight of levulinic acid. The rate of recovery of levulinic acid is 96%, corresponding to the mass of levulinic acid recovered in the distillate relative to the mass of levulinic acid involved in the composition 7. The residue 10 containing the humins and the flux is liquid and viscous at ambient temperature.

These examples show that the presence of a flux makes it possible to significantly increase the recovery rate by mass of levulinic acid compared to conditions in which the flux is not used. This effect is particularly observable when the amount of flux introduced into the residue/flux mixture 7+8 is between 1% and 20% by weight relative to the total weight of the mixture (examples 4 and 5).

1. A process for separating levulinic acid from a composition comprising levulinic acid and humins and optionally compounds having a boiling point lower than that of the levulinic acid, said process comprising:
subjecting the composition to thermal separation in the presence of a flux having a boiling point greater than that of the levulinic acid, to obtain a light fraction containing the levulinic acid and a heavy fraction containing the humins and said flux.

2. The separation process as claimed claim 1, wherein the flux is mixed with said composition and the amount of flux introduced into said mixture is such that the content by mass of the flux in said mixture is between 0.5% and 85% by weight relative to the weight of the mixture.

3. The process as claimed in claim 1, wherein the thermal separation temperature is between 80 and 200° C. and the pressure is between 0.0001 and 0.1 MPa.

4. The process as claimed in claim 1, wherein the flux has a boiling range of between 25° and 620° C. and is of petroleum origin and/or of vegetable origin and/or based on polymers or a mixture thereof.

5. The process as claimed claim 4, wherein the flux is chosen from a petroleum cut chosen from a vacuum gas oil, a heavy oil obtained from a fluidized-bed catalytic cracking, a settling oil, and an unconverted oil originating from a hydrocracker, or is a polyethylene glycol having an average molar mass of greater than or equal to 600 g/mol.

6. The process as claimed in claim 1, wherein the separation step is carried out in at least one distillation column and/or in at least one evaporator.

7. The process as claimed claim 6, wherein the evaporator is a thin film evaporator.

8. The process as claimed in claim 1, wherein, when the composition comprising levulinic acid and humins comprises compounds having a boiling point lower than that of the levulinic acid, said composition is subjected to a preliminary thermal separation so as to separate off the compounds having a boiling point lower than that of the levulinic acid.

9. The process as claimed claim 8, wherein the thermal separation temperature is between 25 and 200° C. and the pressure is between 0.0001 and 0.2 MPa.

10. The process as claimed in claim 8, wherein the separation step is carried out in at least one distillation column and/or in at least one evaporator.

11. The process as claimed in claim 1, wherein the composition comprising levulinic acid and humins is obtained from the synthesis of levulinic acid by hydration of furfuryl alcohol in the presence of an acid catalyst and a solvent.

12. The process as claimed claim 11, wherein the acid catalyst is hydrochloric acid and the solvent is methyl ethyl ketone and/or 1,4-dioxane and/or 1,2-dimethoxyethane.

13. The process as claimed in claim 1, wherein the composition comprising levulinic acid and humins is obtained from the synthesis of levulinic acid by acid hydrolysis of sugar and/or of biomass.

14. The process as claimed in claim 1, wherein the flux has a boiling range of between 25° and 620° C. and is of petroleum origin.

15. The process as claimed in claim 1, wherein the flux has a boiling range of between 250 and 620° C. and is of vegetable origin.

16. The process as claimed in claim 1, wherein the flux has a boiling range of between 25° and 620° C. and is based on polymers.

17. The process as claimed in claim 8, wherein the separation step is carried out in at least one distillation column.

18. The process as claimed in claim 8, wherein the separation step is carried out in at least one evaporator.

19. The process as claimed in claim 16, wherein the flux is based on PEG-600, PEG-800, PEG-1000, PEG-6000, PEG-8000, or mixtures thereof.

20. The process as claimed in claim 1, wherein the flux is PEG 600, a heavy cycle oil, or a vacuum gas oil.
